(19) United States
(12) Patent Application Publication
(10) Pub. No.: US 2023/0112016 A1
(43) Pub. Date: Apr. 13, 2023

(54) GAS-CONCENTRATION DETECTOR SYSTEM

(71) Applicant: Akarsh Aurora, Ashland, MA (US)

(72) Inventor: Akarsh Aurora, Ashland, MA (US)

(21) Appl. No.: 17/957,543

(22) Filed: Sep. 30, 2022

Related U.S. Application Data
(60) Provisional application No. 63/250,768, filed on Sep. 30, 2021.

(51) Int. Cl. G01N 27/12

(52) U.S. Cl. CPC G01N 27/122; G01N 27/129

(57) ABSTRACT

A gas-concentration detector system incorporates a temperature sensor, a humidity sensor, and a gas-concentration detector the accuracy of which deviates as a function of temperature and humidity. Contemporaneous, real-time values of temperature, humidity, and gas concentration of a gas of interest are registered and communicated to a data processing system including a computer memory and a computer processor. Value-deviation data reflective of the degree to which the accuracy of the gas-concentration sensor deviates as a function of temperature and humidity are predetermined and stored in the computer memory. Provided with real-time gas-concentration, temperature, and humidity values, the computer processor executes a value-reconciling algorithm which, based on consultation with the stored value-deviation data, calculates and outputs a refined gas-concentration value more is accurately representative of the actual gas-concentration value within the selected environment in which gas-concentration detection and reporting is desired.

Priority based on Provisional Application Ser. No. 63/250,768 filed Sep. 30, 2021, and titled “GAS-CONCENTRATION DETECTOR SYSTEM” is claimed. Moreover, the entirety of the previous provisional application, including the drawings, is incorporated herein by reference as if set forth fully in the present application.

### BACKGROUND

The use of sensors for detecting the presence and concentrations of specific gases in various environments is a well-established practice. Gas sensors can be categorized according to their operating mechanism (catalytic, electro-chemical, chemFET, resonant, metal oxide semiconductor (MOS), infrared (IR), chromatography, photoionization, chemi-luminescence, etc.).

The metal oxide semiconductor (MOS) gas sensor is a mature innovation and has many advantageous features such as high sensitivity, fast response and recovery, versatile selectivity through operational temperature, and sensor materials as well as good capabilities for low-cost mass production, small size mobile applications, and low power consumption. It has been widely applied in automatic ventilation control systems, household applications, and, more recently, in toxic gas sensor devices. The known sensor technology as such is suitable for measuring various volatile organic compounds and toxic chemical vapors, as well as gases such as NO2, CO, CH4, and H2S.

As with other types of detectors, an MOS gas sensor operates on the principle of environmentally-responsive electrical resistance. More specifically, as the concentration of a gas of interest (also known as a “target gas”) within the environment in which an MOS sensor is situated changes, the electrical resistance across the metal oxide changes as a result of gas absorption by the metal oxide. In much the same way resistance values across a thermistor are correlated to ambient temperatures, resistance values across a metal oxide material are correlated to ambient gas concentrations. The changes in resistance values across the metal oxide in an MOS sensor result from the chemical reduction of oxygen present on the MOS surface by gases of interest, thereby varying the quantity of electrons in the conduction band of the metal oxide material. This resistance drop is reversible and varies depending on the reactivity of sensing materials, the presence of catalyst materials, and working temperature of the sensor.

Along with several advantages, several limitations are associated with MOS gas sensors. Among these are frequently observed drift, poor quality due to reproducibility challenges, narrow dynamic range, and non-linear response. The main consequences of these drawbacks are poor accuracy and poor precision. Typical applications take place in controlled environments, but usage in ambient conditions accompanied by consistent changes in external factors restrict MOS gas sensor employment in measuring outdoor gas concentrations with reliable accuracy.

Two factors that profoundly impact the precision and accuracy of an MOS gas sensor are temperature and humidity. In actuality, the more important humidity factor is “relative humidity,” which, in plain terms, is a measure of how much water vapor is in the air, for example, compared to how much the air could hold at a given temperature. However, throughout the specification and claims, the single term “humidity” is used to indicate humidity-related factors, which could include straight “humidity,” which is the amount of moisture or water present in the air in the form of water vapor without regard to temperature or “relative humidity,” as explained above. Outside of narrow temperature and humidity ranges, indications of gas concentrations derived from MOS gas sensor readings unacceptably deviate from actual ambient gas concentrations, and they do so in a non-linear fashion.

Accordingly, a need exists for a gas concentration detector that, in various embodiments, exploits the advantages associated with MOS sensors while compensating for their associated drawbacks, especially in regard to reliable functionality over temperature and humidity ranges that are broad relative to the capabilities of previous, cost-effective gas detectors.

### SUMMARY

One application envisioned for embodiments of the present invention involves detecting concentrations of climate-impacting gases at specified locations within the earth's atmosphere. Accordingly, gas-concentration detector systems might be deployed near industrial areas, recreational areas, highways, airports, and transportation hubs, by way of non-limiting example. It will be appreciated that temperatures and humidity levels will vary substantially in any given such locale, particularly over a period of weeks, months, or years, and also as a function of geographic location. For instance, New England experiences high temperatures in summer and very cold temperature in winter; a gas concentration detector with a useful temperature range of greater than 100 degrees Fahrenheit may be required. Humidity in New England also fluctuates dramatically over the course of a year, and even over very short timeframes of days or hours.

Based on the preceding, a first illustrative embodiment of a gas-concentration detector system is configured for sensing and reporting concentrations of at least one predetermined gas of interest in a selected environment in which at least one of (i) temperature and (ii) humidity substantially varies over time. The illustrative system includes a gas-concentration sensor responsive to changes in actual gas-concentration values within the selected environment of the at least one gas of interest. The gas-concentration sensor is configured to register, generate, and communicate raw gas-concentration values that correspond to actual gas-concentration values but reflect projected deviations in gas-concentration sensor responsiveness such that, relative to each predetermined actual gas-concentration value, corresponding raw gas-concentration values deviate from that predetermined actual gas-concentration value as a function of at least one of (i) temperature and (ii) humidity within the selected environment.

By way of more concrete example, and as explained in the background, where the gas-concentration sensor is an MOS-based sensor, the electrical resistance across the MOS material will vary with changes in temperature and/or humidity. Because disparate actual gas-concentration values within the environment in which the sensor is situated correlate to disparate resistance values across the MOS material, constant measurement of resistance values across the MOS material functions as registration, generation, and communication of raw gas-concentration values that correspond to actual gas-concentration values. Moreover, because the precision and accuracy of the MOS sensor “drift” as a function of either or both of temperature and humidity, these resistance measurements function as “raw gas-concentration values that correspond to actual gas-concentration values but reflect projected deviations in gas-concentration sensor responsiveness such that, relative to each predetermined actual gas-concentration value, corresponding raw gas-concentration values deviate from that predetermined actual gas-concentration value as a function of at least one of (i) temperature and (ii) humidity within the selected environment.”

In order for the gas-concentration detector system to output meaningful gas-concentration information, the degree of deviation— or “drift”— between a raw gas-concentration value communicated by the gas-concentration sensor and the actual gas-concentration value in the relevant environment to which that raw gas-concentration value corresponds must be determined. As previously noted, this drift is a function of temperature and/or humidity. Accordingly, the first illustrative embodiment includes at least one of a (a) temperature sensor and (b) a humidity sensor configured for registering and communicating, respectively, real-time temperature values and real-time humidity values within the selected environment. “Real-time” in this context means the actual ambient temperature or humidity registered contemporaneously with the registration of corresponding gas-concentration values by the gas-concentration sensor.

Implementation of the gas-concentration detector system further includes a computer processor communicatively linked to the gas-concentration sensor and at least one of the at least one temperature sensor and humidity sensor. The computer processor—or “processing unit”— is configured for receiving, storing, and processing raw gas-concentration values communicated from the gas-concentration sensor and at least one of a temperature value communicated from the temperature sensor and a humidity value communicated from the humidity sensor. While it is envisioned that most implementations will include both (i) at least one temperature sensor AND (ii) at least one humidity sensor, broader aspects of the inventive concept may facilitate the acquisition of only one of temperature and humidity.

In will be appreciated that the drifts of raw gas-concentration values from the corresponding actual gas-concentration values as a function of at least one of temperature and humidity may well be non-linear, particularly over broad ranges of temperature and/or humidity values. Accordingly, each of various implementations includes a computer memory within which is stored value-deviation data reflective of amounts by which raw gas-concentration values generated by the gas-concentration sensor deviate from actual gas-concentration values within the selected environment as a function of at least one of temperature values and humidity values within the selected environment.

In at least one illustrative implementation, pre-correlated, experimentally-determined value-deviation data specific to the gas-concentration sensor embodied in the system may by stored in computer memory to serve functionally as a “look-up table.” For instance, the gas-concentration sensor incorporated into the overall gas-concentration detector system may be situated in a test environment under which humidity and temperature are experimentally varied and raw-gas concentration values are determined relative to that gas-concentration sensor, and others fabricated to the identical specifications so that rapid repeatability in fabrication is achieved. These raw gas-concentration values would then be associated with actual gas-concentration values under the various experimental temperature and humidity conditions as determined by other pre-calibrated instruments within the lab environment. This data— collected over hundreds or perhaps thousands of combinations of temperature and humidity conditions— would then be used to create the value-deviation data that correlates within the look-up table an actual gas-concentration value with a raw gas-concentration value for each experimentally set temperature and/or humidity condition within the useful operating range of the gas-concentration sensor.

In order for the gas-concentration detector system to output meaningful readings that very closely represent actual gas-concentration values, the processing unit is programmed to run a value-reconciling algorithm. The value-reconciling algorithm receives a raw gas-concentration value corresponding to an actual gas-concentration value, consults the stored value-deviation data (e.g., the look-up table), calculates a refined gas-concentration value more accurately representative of the actual gas-concentration value within the selected environment than is the raw gas-concentration value corresponding to that actual gas-concentration value, and outputs the refined gas-concentration value. It should be understood that implementation of an experimentally established look-up table is only one illustrative method of correlating numerous raw gas-concentrations values with actual gas-concentration values to determine a refined gas-concentration value. In alternative implementations, function lines or curves may be mathematically determined from fewer experimentally determined value-deviation data points. These fewer value-deviation data points could then be mathematically fitted to linear or non-linear functions to extrapolate or interpolate where along these functions other raw gas-concentration values versus actual gas-concentration values would reside.

The various components of a gas-concentration detector system within the scope and contemplation of the invention may be embodied within a single, self-contained assembly— or “package”— within which the components are communicatively hardwired. However, expressly within the scope and contemplation of the invention are implementations in which components of the system are disparately located, and wherein the communicative link between at least two of (i) the gas-concentration sensor, (ii) the temperature sensor, (iii) the humidity sensor, (iv) the computer processor, and (v) the computer memory is wireless. In either case, in accordance with at least one implementation, the refined gas-concentration value caused to be outputted is by the computer processor is further communicated least one of (i) to a programmed machine other than the gas-concentration detector system and (ii) in a human-comprehensible format through a machine-to-human interface associated with the gas-concentration detector system (e.g., a display screen)

Representative, non-limiting implementations are more completely described and depicted in the following detailed description and the accompanying drawings.

### BRIEF DESCRIPTION OF THE DRAWINGS

FIG. 1 is a schematic representation of an illustrative gas-concentration detection system; and

FIG. 2 is a graphical representation modeling RS/Ro vs. temperature curves at various relative humidities.

### DETAILED DESCRIPTION

The following description of apparatus for and methods of sensing and reporting concentrations of at least one predetermined gas of interest in a selected environment in which temperature and humidity vary over time is illustrative in nature and is therefore not intended to limit the scope of the invention or its application of uses. Accordingly, the various implementations, aspects, versions and embodiments described in the summary and detailed description are in the nature of non-limiting examples falling within the scope of the appended claims and do not serve to restrict the maximum scope of the claims.

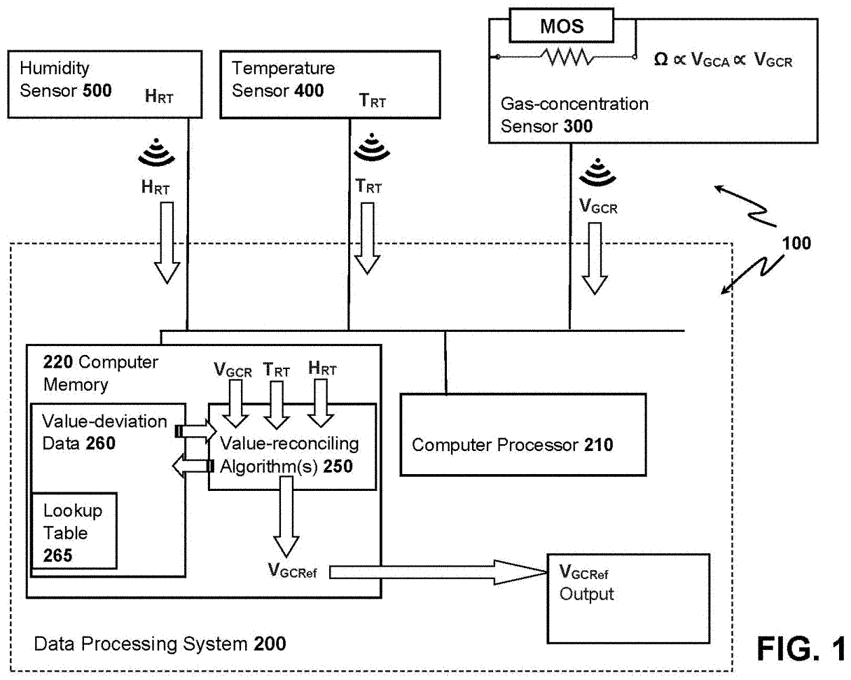

Referring to FIG. 1, the architecture of an illustrative gas-concentration detector system 100 is schematically represented and includes (i) a data processing system 200 including a computer processor 210 and a computer memory 220; (ii) a gas-concentration sensor 300, (iii) a temperature sensor 400, and (iv) a humidity sensor 500. Each of the gas-concentration sensor 300, the temperature sensor 400, and the humidity sensor 500 is communicatively linked to the data processing system 200, either directly or through intermediate data-relaying components (not shown). Moreover, the communicative link(s) between the data-processing system 200 and each of the gas-concentration sensor 300, temperature sensor 400, and humidity sensor 500 may be hardwired or wireless, which is indicated in the schematic by showing the communicative links as both unbroken connecting lines and a plurality of concentric arcs indicative of wave fronts ubiquitously understood as an indication of a wireless link.

The gas-concentration sensor 300 is configured to register, generate, and communicate raw gas-concentration values V_{GCR} that correspond to actual gas-concentration values V_{GCA} but reflect projected deviations in gas-concentration sensor responsiveness such that, relative to each predetermined actual gas-concentration value V_{GCA}, corresponding raw gas-concentration values V_{GCR} deviate from that predetermined actual gas-concentration value V_{GCA} as a function of at least one of (i) temperature and (ii) humidity (e.g., actual moisture content or relative humidity, as described in the summary) within the selected environment.

In each of various implementations, including the one depicted in FIG. 1, the gas-concentration sensor 300 is an MOS-based sensor in which the electrical resistance across the MOS material varies with changes in temperature and/or humidity. Because disparate actual gas-concentration values V_{GCA} within the environment in which the gas-concentration sensor 300 is situated correlate to disparate resistance values Ω across the MOS material, constant measurement of resistance values across the MOS material functions “as registration, generation, and communication of raw gas-concentration values V_{GCR} that correspond to actual gas-concentration values V_{GCA}.” Moreover, because the precision and accuracy of the MOS sensor deviates as a function of either or both of temperature and humidity, these resistance measurements function as “raw gas-concentration values V_{GCR} that correspond to actual gas-concentration values V_{GCA} but reflect projected deviations in gas-concentration sensor responsiveness such that, relative to each predetermined actual gas-concentration value V_{GCA}, corresponding raw gas-concentration values V_{GCR} deviate from that predetermined actual gas-concentration value V_{GCA} as a function of at least one of (i) temperature and (ii) humidity within the selected environment.”

In order for the gas-concentration detector system 100 to output meaningful gas-concentration information, the degree of deviation between a raw gas-concentration value V_{GCR} communicated by the gas-concentration sensor 300 and the actual gas-concentration value V_{GCA} in the relevant environment to which that raw gas-concentration value V_{GCA} corresponds must be determined. As previously noted, this deviation is most frequently a function of both temperature and humidity within the selected environment at the time a raw gas-concentration value V_{GCR} is registered by the gas-concentration sensor 300 (i.e., in “real time). Accordingly, the illustrative embodiment of FIG. 1 includes both a (a) temperature sensor 400 configured for registering and communicating to the data processing system 200 real-time temperature values T_{RT} and (b) a humidity sensor 500 configured for registering and communicating to the data processing system 200 real-time humidity values H_{RT} within the selected environment.

The computer processor 210 is configured for receiving, storing, and processing is raw gas-concentration values V_{GCR} communicated from the gas-concentration sensor 300 as well as temperature values T_{RT} communicated from the temperature sensor 400 and humidity values H_{RT} communicated from the humidity sensor 500. More specifically, the computer processor 210 receives and synthesizes raw gas-concentration values V_{GCR}, temperature values T_{RT}, and humidity values H_{RT}, and, by running a value-reconciling algorithm 250, generates and outputs corresponding refined gas-concentration values V_{GCRef}. There are various methods by which raw gas-concentration values V_{GCR} can be algorithmically correlated to refined gas-concentration values V_{GCRef} based on real-time temperature and humidity values T_{RT} and H_{RT}, illustrative, non-limiting examples of which are briefly described below.

In each of various implementations, there is stored in the computer memory 220 value-deviation data 260 reflective of amounts by which raw gas-concentration values V_{GCR} generated and communicated by the gas-concentration sensor 300 deviate from actual gas-concentration values V_{GCA} as a function of real-time temperature and humidity values T_{RT} and H_{RT}. In one version, pre-correlated, experimentally-determined value-deviation data 260 specific to the gas-concentration sensor 300 embodied in the detector system 100 may by stored in computer memory 220 to serve functionally as a look-up table 265. As described in the summary, the gas-concentration sensor 300 ultimately incorporated into the overall gas-concentration detector system 100 may first be situated in a test environment under which humidity and temperature are experimentally varied and raw-gas concentration values V_{GCA} are determined relative to that gas-concentration sensor 300, and other gas-concentration sensors fabricated to identical specifications. These raw gas-concentration values V_{GCR} would then be associated with actual gas-concentration values V_{GCA} under the various experimental temperature and humidity conditions as determined by other pre-calibrated instruments within the lab environment. That is, the raw gas-concentration values V_{GCR} would be pre-associated with actual gas-concentration values V_{GCA} and corresponding, is experimentally set real-time temperature and humidity values T_{RT} and H_{RT} in order to construct the value-deviation data 260 (e.g., the lookup table 265).

Regardless of how the value-deviation data 260 is created, the value-reconciling algorithm 250 receives a real-time temperature value T_{RT}, a real-time humidity value H_{RT}, and a raw gas-concentration value V_{GCR} corresponding to an actual gas-concentration value V_{GCA}; consults the stored value-deviation data 260; generates a refined gas-concentration value V_{GCRef} more accurately representative of the actual gas-concentration value V_{GCA} within the selected environment than is the raw gas-concentration value V_{GCR} corresponding to that actual gas-concentration value V_{GCA}; and outputs the refined gas-concentration value V_{GCRef}.

In alternative implementations, function lines or curves may be mathematically determined from fewer experimentally determined value-deviation data points in order to create value-deviation data 260. These fewer value-deviation data points could then be mathematically fitted to linear or non-linear functions to extrapolate or interpolate where along these functions other raw versus actual gas-concentration values would reside.

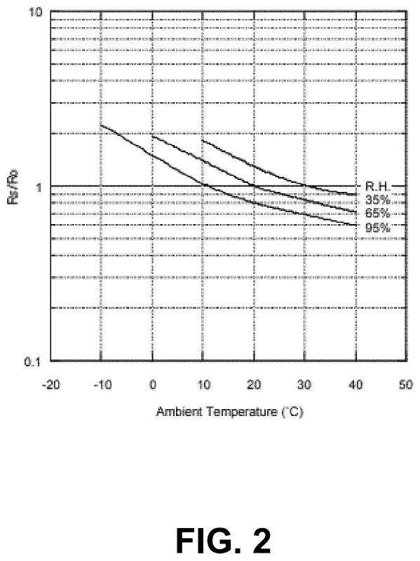

FIG. 2 is a graphical representation of various experimentally determined “correlation curves” plotted in response to varying, experimentally-controlled environmental conditions. More specifically, sensitivity curves are plotted for three disparate real-time humidity values H_{RT} in the control environment. For the example shown, the illustrative real-time humidity values H_{RT} are relative humidities (R.H.) of 35%, 65%, and 95%. Each “humidity curve” relates a resistance ratio of an MOS-based gas-concentration sensor 300 (y-axis) to disparate real-time temperature values T_{RT} (x-axis) in the control/calibration environment. The resistance ratio is expressed as R_{S}/Rₒ, where the value R_{S} represents the variable resistance of the gas-concentration sensor 300 at a particular real-time humidity value H_{RT} at various temperature values T_{RT} in “fresh air” and Rₒ is the resistance of the gas-concentration sensor 300 at a predetermined set of reference/calibration values (e.g. sensor resistance in “fresh air” at 20° C. and 65% relative humidity). By “fresh air” is meant air as it naturally occurs in an environmental totally devoid of pollutants and/or abnormal concentrations of gases the gas-concentration sensor 300 is designed to detect. The experimental data collected under test conditions at various fixed real-time humidity values H_{RT} is used to create value-deviation data 260 in a gas-concentration detector system 100 such as that schematically depicted in FIG. 1.

Out in the field, where real-time gas-concentration detection is to occur, the computer processor 210 accesses raw gas-concentration values V_{GCR} and real-time temperature and humidity values T_{RT} and H_{RT} as they are communicated to the data processing system 200 by the gas-concentration sensor 300, the temperature sensor 400, and the humidity sensor 500. Employing power regression, a new correlation curve is drawn by “squeezing” for the measured real-time humidity value H_{RT}. The ambient real-time temperature value T_{RT} is plugged into the approximation equation to yield the corresponding sensitivity ratio for the exact real-time conditions present in the environment. The MOS sensor resistance in fresh air at test conditions is divided by the newly acquired ratio to render a corrected actual sensor resistance which accounts for temperature and humidity changes across time.

The foregoing is considered to be illustrative of the principles of the invention. Furthermore, since modifications and changes to various aspects and implementations will occur to those skilled in the art without departing from the scope and spirit of the invention, it is to be understood that the foregoing does not limit the invention as expressed in the appended claims to the exact constructions, implementations and versions shown and described.

What is claimed is:

1. A gas-concentration detector system for sensing and reporting concentrations of at least one predetermined gas of interest in a selected environment in which temperature and humidity vary over time, the detector system comprising:
a gas-concentration sensor responsive to
changes in actual gas-concentration values within the selected environment of the at least one gas of interest, the gas-concentration sensor being configured to generate and communicate raw gas-concentration values that correspond to actual gas-concentration values but reflect projected deviations in gas-concentration sensor responsiveness such that, relative to each predetermined actual gas-concentration value, corresponding raw gas-concentration values deviate from that predetermined actual gas-concentration value as a function of at least one of (i) temperature and (ii) humidity within the selected environment;
at least one of a (a) temperature sensor and (b) a humidity sensor configured for registering and communicating, respectively, temperature values and humidity values within the selected environment;
a central computer processor communicatively linked to the gas-concentration sensor and the at least one of the at least one temperature sensor and humidity sensor, and configured for receiving, storing, and processing raw gas-concentration values communicated from the gas-concentration sensor and at least one of a temperature value communicated from the temperature sensor and a humidity value communicated from the humidity sensor; and
a computer memory within which is stored value-deviation data reflective of amounts by which raw gas-concentration values generated by the gas-concentration sensor deviate from actual gas-concentration values with the selected environment as a function of at least one of temperature values and humidity values within the selected environment;
wherein the computer processor is programmed to run a value-reconciling algorithm that receives a raw gas-concentration value corresponding to an actual gas-concentration value, consults the stored value-deviation data, calculates a refined gas-concentration value more accurately representative of the actual gas-concentration value within the selected environment than is the raw gas-concentration value corresponding to the that actual gas-concentration value, and output the refined gas-concentration value.

2. The gas-concentration detector system of claim 1 wherein, relative to each predetermined actual gas-concentration value, corresponding raw gas-concentration values deviate from that predetermined actual gas-concentration value as a function of both temperature and humidity within the selected environment.

3. The gas-concentration detector system of claim 2 wherein the deviation of raw gas-concentration values from the corresponding actual gas-concentration value as a function of at least one of temperature and humidity is non-linear.

4. The gas-concentration detector system of claim 3 wherein the deviation of raw gas-concentration values from the corresponding actual gas-concentration value as a function of both temperature and humidity is non-linear.

5. The gas-concentration detector system of claim 4 wherein the refined gas-concentration value caused to be outputted by the computer processor is further communicated least one of (i) to a programmed machine other than the gas-concentration detector system and (ii) in a human-comprehensible format though a machine-to-human interface associated with the gas-concentration detector system.

6. The gas-concentration detector system of claim 2 wherein the refined gas-concentration value caused to be outputted by the computer processor is further communicated least one of (i) to a programmed machine other than the gas-concentration detector system and (ii) in a human-comprehensible format though a machine-to-human interface associated with the gas-concentration detector system.

7. The gas-concentration detector system of claim 1 wherein the refined gas-concentration value caused to be outputted by the computer processor is further communicated least one of (i) to a programmed machine other than the gas-concentration detector system and (ii) in a human-comprehensible format though a machine-to-human output interface associated with the gas-concentration detector system.

8. The gas-concentration detector system of claim 7 wherein the communicative link between at least two of (i) the gas-concentration sensor, (ii) the temperature sensor, (iii) the humidity sensor, (iv) the central computer processor, (v) the computer memory, (vi) the programmed machine other than the gas-concentration detector system, and (vii) the machine-to-human output interface is wireless.

9. The gas-concentration detector system of claim 1 wherein the communicative link between at least two of (i) the gas-concentration sensor, (ii) the temperature sensor, (iii) the humidity sensor, (iv) the central computer processor, and (v) the computer memory is wireless.

10. The gas-concentration detector system of claim 1 wherein the gas-concentration sensor is a metal oxide semiconductor sensor.

11. The gas-concentration detector system of claim 10 wherein, relative to each predetermined actual gas-concentration value, corresponding raw gas-concentration values deviate from that predetermined actual gas-concentration value as a function of both temperature and humidity within the selected environment.

12. The gas-concentration detector system of claim 11 wherein the deviation of raw gas-concentration values from the corresponding actual gas-concentration value as a function of at least one of temperature and humidity is non-linear.
